(19) United States
(12) Patent Application Publication
(10) Pub. No.: US 2012/0214771 A1
(43) Pub. Date: Aug. 23, 2012

(54) COMPOSITIONS FOR TREATMENT OF CARDIOMETABOLIC DISORDERS

(76) Inventor: Fontini Sampalis, Laval (CA)

(21) Appl. No.: 13/305,254

(22) Filed: Nov. 28, 2011

Related U.S. Application Data
(63) Continuation-in-part of application No. 13/189,714, filed on Jul. 25, 2011.
Application No. 13/189,714 is a continuation of application No. 10/485,094, filed on Jul. 13, 2004, now Pat. No. 8,030,348.
(60) Provisional application No. 60/307,842, filed on Jul. 27, 2001.

(51) Int. Cl. A61K 31/661; A61P 9/00; A61P 9/10; A61P 3/06; A61P 7/00; A61P 9/12; A61P 9/04; A61P 9/06; A61P 3/10; A61P 5/48; A61P 3/08; A61P 25/00; A61P 13/12; A61P 1/16; A61P 7/02; A61P 3/00

(52) U.S. Cl. 514/114; 514/121

(57) ABSTRACT

The present invention provides for methods of preventing or improving cardiometabolic disorders/metabolic disorders, compositions and pharmaceutical compositions comprising therapeutically effective amount of therapeutic phospholipid compositions and therapeutically effective amount of one or more lipid modifying agents, including statins.

### PRIORITY

The present application is a continuation in part of pending U.S. patent application Ser. No. 13/189,714, filed Jul. 25, 2011, which is a continuation of U.S. patent application Ser. No. 10/485,094 (now U.S. Pat. No. 8,030,348 issued Oct. 4, 2011), filed Jul. 13, 2004, which is the §371 National Phase of International Application No. PCT/CA02/01185, filed Jul. 29, 2002, which claims the benefit of priority to U.S. Provisional Application Ser. No. 60/307,842, filed Jul. 27, 2001. All of these documents are incorporated herein by reference in their entireties.

### FIELD OF THE INVENTION

The invention relates to methods, compositions, and pharmaceutical compositions for treating or preventing cardiometabolic disorders.

### BACKGROUND

Cardiovascular disease is a leading cause of morbidity and mortality, particularly in developed areas such as the United States, Canada, and Western Europe. Hoyert D. L., et al., National Vital Statistics Reports, 2005. Among cardiovascular diseases, Chronic Heart Failure (CHF) is the most common medical condition afflicting the Western world. The major cause of CHF is myocardial infarction or the death of heart muscle during a heart attack, caused by coronary atherosclerosis. Coronary atherosclerosis refers to the hardening and narrowing of the coronary arteries. When coronary arteries are narrowed or blocked by atherosclerosis, they cannot deliver an adequate amount of blood to the heart muscle. Disease caused by the lack of blood supply to heart muscle is called coronary heart disease (CHD). Coronary heart diseases are characterized by heart attacks, sudden unexpected death, chest pain, abnormal heart rhythms, and heart failure due to weakening of heart muscle.

One of the risk factors for developing coronary atherosclerosis is elevated blood cholesterol. A person's cholesterol level is affected, among others, by age, sex, heredity and diet. Cholesterol is vital for healthy cells. Thus, the body does not rely solely on a dietary source, but produces its own cholesterol. However, if the body accumulates too much cholesterol, the cholesterol will deposit on the walls of arteries, which can damage or block the arteries, and cause a heart attack. A total cholesterol level in excess of 225-250 mg/dl is associated with significant elevation of the risk of CHD. Cholesteryl esters are a major component of atherosclerotic lesions and the major storage form of cholesterol in arterial wall cells. Formation of cholesteryl esters is also a key step in the intestinal absorption of dietary cholesterol. Thus, inhibition of cholesteryl ester formation and reduction of serum cholesterol is likely to inhibit the progression of atherosclerotic lesion formation, decrease the accumulation of cholesterol esters in the arterial wall, and block the intestinal absorption of dietary cholesterol.

Lowering cholesterol is important for nearly all subjects, including, for example, younger, middle-aged, and older adults, and people with or without heart disease and/or stroke. Lowering high cholesterol levels lessens the risk for developing heart disease and reduces the chance of a heart attack or death by heart disease. Stone, N J, Endocrinol Metab Clin North Am., 1990, 19(2):321-44); Chapman, M. J. Curr. Med. Res. Opin., 2005, 21 (Suppl 6):S17-22).

Medications that reduce blood cholesterol levels include, but are not limited to, various lipid modifying agents including cholesterol sequestration drugs; triglyceride-lowering drugs; and cholesterol pathway blockers (statins). Cholesterol sequestration drugs (resins), such as cholestyramine (Questran) and colestipol (Colestid), are used to lower cholesterol indirectly by binding with bile acids in the intestinal tract. The liver makes bile acids from cholesterol and therefore resins sequester bile acids which induce the production of more bile acids and a reduction in cholesterol. Schmitz, et al. T. Vascul. Pharmacol., 2006, 44(2):75-89; Schmitz, et al. Clin. Chem. Lab. Med., 2003, 41(4):581-9. Triglyceride-lowering drugs include fibrates, such as gemfibrozil (Lopid) and fenofibrate (Tricor), and the vitamin niacin (nicotinic acid), which reduce triglyceride production and remove triglycerides from circulation. They can also increase HDL. Gotto, A M. J. Am. Heart J., 2002, 144(6):S33-42; Miller, et al. Clin. Chim. Acta., 1988, 178(3):251-9.

Statins are competitive inhibitors of HMG-CoA reductase, a key enzyme in the cholesterol biosynthesis pathway. This inhibition depletes cholesterol in liver cells, which causes the liver cells to remove cholesterol from the blood. Rodenburg, J. et al. Pediatr. Endocrinol. Rev., 2004, 2(Suppl 1):171-80; Gylling, H. et al. Curr. Opin. Investig. Drugs., 2006 March, 7(3):214-8. Statins can reduce LDL cholesterol by up to 40 percent. Statins may also help the body reabsorb cholesterol from plaques that accumulate on the walls of the arteries, thus making them less likely to cause complications such as heart attack or stroke. Statins include cerivastatin, atorvastatin, simvastatin, pravastatin, fluvastatin, lovastatin, rosuvastatin, and pitavastatin. Stroes, et al. Curr. Med. Res. Opin., 2005, 21(Suppl 6):S9-16.

However, statin treatment is limited. For instance, side effects can include myositis, headache, rash, angioedema, gastrointestinal effects, myopathy, rhabdomyolysis and altered liver functions. In addition, these drugs should not be used in patients with renal failure or in patients with compromised liver function. Taylor, et al., 2003. “Octacosanol in human health.” Nutrition. 19: 192-195. Further, many physiologic nutrients of small molecular weight are produced from the mevalonate pathway that generates the “isoprenoid pool” (IP) products. Geraniol (G), farnesol (F), and geranyl geraniol (GG) are examples of IP products containing two, three, and four repeating units of five-carbon isoprenes, respectively. However, statins inhibit mevalonate (e.g., one isoprene) at the onset of the formation of the first isoprene, and therefore inhibit all subsequent IP products, including GG. This depletion and deprivation of GG can produce secondary, but clinically significant, side effects of DL-starved cranial nerve damage and defects typified by neurological dysfunctions (e.g., taste alteration/loss, lack coordination, facial paresis, memory loss, vertigo, peripheral neuropathy, and peripheral nerve palsy). Accordingly, statins are limited by negative health effects; other agents that can regulate the cholesterol biosynthesis, absorption and metabolism have similar limitations.

In July 2004, the National Cholesterol Education Program (NCEP) published a paper entitled “Implications of Recent Clinical Trials for the National Cholesterol Education Program Adult Treatment Panel III Guidelines,” which updated certain elements of the “Adult Treatment Panel III (ATP III)” cholesterol guidelines released in 2001. For high-risk patients, individuals who have coronary heart disease (CHD) or disease of the blood vessels to the brain or extremities, or diabetes, or multiple (2 or more) risk factors that give them a greater than 20 percent chance of having a heart attack within 10 years, the ATP III update recommends an LDL less than 100 mg/dL with a therapeutic option to set the goal at an LDL less than 70 mg/dL for very high-risk patients, those who have had a recent heart attack, or those who have cardiovascular disease combined with either diabetes, or severe or poorly controlled risk factors (such as continued smoking), or metabolic syndrome (a cluster of risk factors associated with obesity that includes high triglycerides and low HDL cholesterol). The ATP III update also recommends consideration of drug treatment in addition to lifestyle therapy for LDL levels 100 mg/dL or higher in high-risk patients, and characterizes drug treatment as optional for LDL less than 100 mg/dL. For moderately high-risk patients, individuals who have multiple (2 or more) CHD risk factors together with a 10-20 percent risk for a heart attack within 10 years, the ATP III update recommends an LDL of less than 130 mg/dL. There is a therapeutic option to set the treatment goal at an LDL less than 100 mg/dL, and to use drug treatment if the LDL is 100-129 mg/dL. The ATP III update advises that the intensity of LDL-lowering drug treatment in high-risk and moderately high-risk patients be sufficient to achieve at least a 30 percent reduction in LDL levels. But, patients suffering from severe hypercholesterolemia may be unable to reach the new goals for LDL and HDL described above. For example, a large number of patients may be unable to attain LDL levels less than 70 using maximally tolerated current methodologies.

Thus, there is a need to develop methods, agents, and compositions for treating hyperlipidemia and/or hypercholesterolemia that are efficacious in lowering serum cholesterol and LDL, increasing HDL serum levels, preventing coronary heart disease, and/or treating diseases associated with hyperlipidemia and/or hypercholesterolemia, without the serious side-effects and patient variability associated with the known treatments. There is also a need for the development of methods and therapeutic agents that are efficacious in lowering serum cholesterol levels in combination with known therapeutic agents, for example with statins. Such a combination therapy allows the use of statins such as atorvastatin (Lipitor) in lower dosages, with minimal or no side effects.

### SUMMARY OF THE INVENTION

Accordingly, the present invention provides for improved methods, compositions, and pharmaceutical compositions for treating or preventing cardiometabolic disorders.

In one aspect, the invention provides a method of treating a cardiometabolic disorder/metabolic syndrome, the method comprising administering to a subject in need thereof a therapeutically effective amount of a lipid modifying agent and a therapeutically effective amount of a composition comprising a phospholipid comprises a glycerol backbone wherein one of the sn-1 and sn-2 positions is selected from a docosahexaenoic acid (DHA) or an eicosapentaenoic acid (EPA) residue and the other sn-1 and sn-2 position is a fatty acid.

In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In a specific embodiment, the omega 3-fatty acid or a fatty acid that is metabolized to an omega 3-fatty acid is selected from the group consisting of hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In yet another embodiment, the phospholipid is a compound of Formula (I):

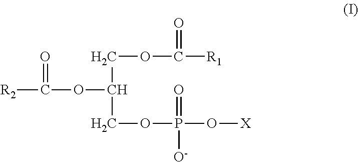

wherein R1 and R2, each together with the respective carboxyl groups to which each is attached, each independently represent a docosahexaenoic acid (DHA) or an eicosapentaenoic acid (EPA) residue, and X is —CH₂CH₂NH₃, —CH₂CH₂N(CH₃)₃, or

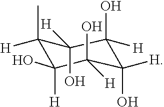

In another embodiment, the cardiometabolic disorder/metabolic syndrome is selected from atherosclerosis, dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoprotenemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel, and diabetic autonomic neuropathy.

In still another embodiment, the methods described herein further comprise preventing, reducing or treating elevated cholesterol levels, atherosclerosis, hyperlipidemia, hypercholesterolemia, cardiovascular events and disease including coronary events and cerebrovascular events, and coronary artery disease and/or cerebrovascular disease in a patient in need thereof.

In a further embodiment, the lipid modifying agent is a statin. In a specific embodiment, the statin is selected from the group consisting of cerivastatin, atorvastatin, simvastatin, pravastatin, fluvastatin, lovastatin, rosuvastatin, and pitavastatin. In another specific embodiment, the statin is present in less than about 10-50 mg/d. In some embodiments, the amount of the statin is less than the amount of lipid modifying agent administered without the composition comprising a phospholipid.

In another aspect, the invention provides a pharmaceutical composition comprising a therapeutically effective amount of a lipid modifying agent, selected from the group consisting of statins, ezetimibe, fibrates, niacinamide, niacin, and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In a specific embodiment, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the phospholipid composition is a compound of Formula (I):

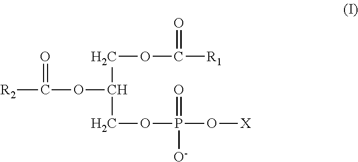

wherein R1 and R2, each together with the respective carboxyl groups to which each is attached, each independently represent a docosahexaenoic acid (DHA) or an eicosapentaenoic acid (EPA) residue, and X is —CH₂CH₂NH₃, —CH₂CH₂N(CH₃)₃, or

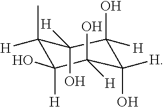

### BRIEF DESCRIPTION OF THE DRAWINGS

FIG. 1. A double blind prospective randomized clinical study in patients with hypercholerolemia who received either 1.0-1.5 g/daily NKO® or 3.0 g/daily fish oil.

FIG. 2. A double blind prospective randomized clinical study in patients diagnosed with hyperlipidemia who were on a 10 mg dose of statin regimen for at least 6 months, who received either 1.0-1.5 g/daily NKO® or 3.0 g/daily fish oil.

FIG. 3. Total cholesterol levels (absolute change) in a double blind prospective randomized clinical study in patients diagnosed with hyperlipidemia who were on a 10 mg dose of statin regimen for at least 6 months, who received either 10 mg dose of statin or a combination therapy of 10 mg dose of statin and 1.0-1.5 g/daily NKO®.

FIG. 4. HDL-C (absolute change) in a double blind prospective randomized clinical study in patients diagnosed with hyperlipidemia who were on a 10 mg dose of statin regimen for at least 6 months, who received either 10 mg dose of statin or a combination therapy of 10 mg dose of statin and 1.0-1.5 g/daily NKO®.

FIG. 5. LDL-C (absolute change) in a double blind prospective randomized clinical study in patients diagnosed with hyperlipidemia who were on a 10 mg dose of statin regimen for at least 6 months, who received either 10 mg dose of statin or a combination therapy of 10 mg dose of statin and 1.0-1.5 g/daily NKO®.

FIG. 6. Fatal cardiovascular event absolute risk in a double blind prospective randomized clinical study in patients diagnosed with hyperlipidemia who were on a 10 mg dose of statin regimen for at least 6 months, who received either 10 mg dose of statin or a combination therapy of 10 mg dose of statin and 1.0-1.5 g/daily NKO®.

FIG. 7 shows the effect of the combination therapy of 10 mg/day dose of statin and either 1.0 g/daily or 3 g/daily of NKO®, 10 mg/day dose of statin and 3.0 g/daily fish oil, or 10 mg/day dose of statin and placebo on HDL levels in subjects over 61 years old.

FIG. 8 shows the LDL reducing effect of the combination therapy (10 mg dose of statin and 1.0-1.5 g/daily NKO®) on levels of LDL in subjects after 90 days of treatment.

FIG. 9 shows the HDL increasing effect of the combination therapy (10 mg dose of statin and 1.0-1.5 g/daily NKO®) on levels of HDL in subjects after 90 days of treatment.

FIG. 10. Total cost and cost effectiveness of combination treatment versus fish oil, fish oil combinations, statin alone, and placebo.

### DETAILED DESCRIPTION

All publications, patents and patent applications, including any drawings and appendices herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

### DEFINITIONS

“Enhancing activity” means, when co-administered, the lipid modifying agent is administered at lower effective dosage than the effective dosage of statin if administered alone. At the lower effective dosages, when co-administered with phospholipid compositions, the lipid modifying agent provide equal or superior efficacy than higher effective dosages administered alone.

“Cardiometabolic disorder(s),” “Cardiac diseases” and “metabolic disorder(s)” refer to atherosclerosis, arteriosclerosis, coronary heart (crotid artery) disease (CHD or CAD), acute coronary syndrome (or ACS), valvular heart disease, aortic and mitral valve disorders, arrhythmia/atrial fibrillation, cardiomyopathy and heart failure, angina pectoris, acute myocardial infarction (or AMI), hypertension, orthostatic hypotension, shock, embolism (pulmonary and venous), endocarditis, diseases of arteries, the aorta and its branches, disorders of the peripheral vascular system (peripheral arterial disease or PAD), Kawasaki disease, congenital heart disease (cardiovascular defects) and stroke (cerebrovascular disease), dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoprotenemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

The term “coronary artery disease” (CAD) as employed herein refers to diseases including atherosclerosis of the coronary arteries, previous myocardial infarction, ischemia, angina pectoris, and/or heart failure.

The term “cerebrovascular disease” as employed herein refers to diseases including atherosclerosis of the intracranial and/or extracranial arteries, cerebral infarction, cerebral thrombosis, cerebral ischemia, stroke, and/or transient ischemic attacks.

The terms “cardiovascular event(s)” and “cardiovascular disease” as employed herein refer to coronary and/or cerebrovascular event(s) and disease including primary myocardial infarction, secondary myocardial infarction, myocardial ischemia, angina pectoris (including unstable angina), congestive heart failure, sudden cardiac death, cerebral infarction, cerebral thrombosis, cerebral'ischemia, transient ischemic attack, and the like.

“Fatty acids” are an important component of nutrition. Fatty acids (also described as “free acids” or “free fatty acids”) are carboxylic acids and are classified based on the length and saturation characteristics of the carbon chain. Short chain fatty acids have about 2 to about 5 carbons and are typically saturated. Medium chain fatty acids have from about 6 to about 14 carbons and are also typically saturated. Long chain fatty acids have from about 15 to about 24 or more carbons and may also be saturated or unsaturated. In longer fatty acids there may be one or more points of unsaturation, giving rise to the terms “monounsaturated” and “polyunsaturated,” respectively.

“Long chain polyunsaturated fatty acids” (or “LC-PUFAs”) are categorized according to the number and position of double bonds in the fatty acids according to an accepted nomenclature that is well-known to those of ordinary skill in the art. There are two series or families of LC-PUFAs, depending on the position of the double bond closest to the methyl end of the fatty acid: the n-3 series contains a double bond at the third carbon, while the n-6 series does not have a double bond until the sixth carbon. Thus, arachidonic acid (AA or ARA) has a chain length of 20 carbons and 4 double bonds beginning at the sixth carbon. As a result, it is referred to as “20:4 n-6”. Similarly, docosahexaenoic acid (DHA) has a chain length of 22 carbons with 6 double bonds beginning with the third carbon from the methyl end and is thus designated “22:6 n-3”. Another important LC-PUFA is eicosapentaenoic acid (EPA) which is designated (20:5 n-3). The terms “n-3” and “omega-3” are used interchangeably

The “essential fatty acids” (EFAs) are of two types, the n-3 (or omega-3) series derived from alpha-linolenic acid and the n-6 (or omega-6) series derived from linoleic acid.

Pharmaceutical compositions and medicaments may be described as mixtures of two or more components “by volume,” which is herein defined as the volume due to one component divided by the volume of all components of the composition. This ratio may be converted to or reported as a percentage of the total composition volume. Such a quantity may also be indicated by “v/v” or “percent v/v.” Similarly, the phrases “by weight” and “by mass” describe the weight or mass due to one component divided by the weight or mass of all components of the composition. This ratio may be converted to or reported as a percentage of the total composition weight or mass. Such a quantity may also be indicated by “w/w,” “mass percent,” or “percent w/w.”

The terms “pharmaceutical composition” and “formulation” are used interchangeably throughout the specification and claims.

According to the present invention, the mass of a salt of a statin is measured with respect to the mass of the free form. For example, an 80 mg amount of a salt of a statin refers to 80 mg of free form statin, without the mass of the cation being included.

“Statin” as used herein includes, but is not limited to, pravastatin, fluvastatin, atorvastatin, lovastatin, simvastatin, rosuvastatin, and cerivastatin. Statins may be in the form of a salt, hydrate, solvate, polymorph, or a co-crystal. Statins may also be in the form of a hydrate, solvate, polymorph, or a co-crystal of a salt. Statins may also be present in the free acid or acetone form. In addition, the term “statin” used herein includes synthetic and natural analogs of pravastatin, fluvastatin, atorvastatin, lovastatin, simvastatin, rosuvastatin, cerivastatin, and any other statins known by those skilled in the art.

The term “therapeutic phospholipid composition” and “therapeutic phospholipid compositions” as used herein refer to the therapeutic phospholipid compositions comprising compounds of the present invention.

The articles “a” and “an” are used in this disclosure to refer to one or more than one (i.e., to at least one) of the grammatical object of the article. By way of example, “an element” means one element or more than one element.

The term “about” when used in this disclosure along with a recited value means the value recited and includes the range of + or −5% of the value, or of + or −10% of the value, or of + or −15% of the value, or of + or −20% of the value, or of + or −25% of the value, or of + or −50% of the value. The recited value “about 0%” as used herein means that the detectable amount may be less than one part per thousand.

The term “fatty acid” or “fatty acid residue” as used herein means a carboxylic acid with a long unbranched aliphatic chain, which is either saturated or unsaturated. Saturated fatty acids have the general formula CₙH₂ₙ+1 COOH. Examples of saturated fatty acids include but are not limited to: propanoic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, triacontanoic acid, henatriacontanoic acid, dotriacontanoic acid, tritriacontanoic acid, tetratriacontanoic acid, pentatriacontanoic acid, and hexatriacontanoic acid. An unsaturated fat is a fat or fatty acid in which there are one or more double bonds in the fatty acid chain. A fat molecule is monounsaturated if it contains one double bond and polyunsaturated if it contains more than one double bond. Examples of unsaturated fatty acids include but are not limited to: myristoleic acid, palmitoleic acid, sapienic acid, oleic acid, linoleic acid, α-linolenic acid, arachidonic acid, eicosapentaenoic acid (EPA), erucic acid, docosahexaenoic acid (DHA), and docosapentaenoic acid.

A “subject” is a mammal, e.g., a human, mouse, rat, guinea pig, dog, cat, horse, cow, pig, or non-human primate, such as a monkey, chimpanzee, baboon or rhesus.

Representative “pharmaceutically acceptable salts” include, by way of non-limiting example, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disorder” as used herein refers to disorders, diseases and syndromes involving dyslipidemia or hyperlipidimia, and the terms metabolic disorder, metabolic disease, and metabolic syndrome are used interchangeably herein.

The term “treating,” with regard to a subject, refers to improving at least one symptom of the subject's disorder. Treating can be curing, improving, or partially ameliorating the disorder.

The term “disorder” is used in this disclosure to mean, and is used interchangeably with, the terms disease, condition, abnormal physiological condition, abnormal levels of one or more biomarkers, or illness, unless otherwise indicated.

The term “administer,” “administering,” or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The terms “an effective amount,” “therapeutic effective amount,” or therapeutically effective amount” shall mean an amount or concentration of a composition according to the present invention which is effective in producing a desired result within the context of its administration or use, including, for example, lowering LDL-C levels, increasing HDL-C levels, or lowering LDL-C levels and increasing HDL-C levels. Thus, the term “effective amount” is used throughout the specification to describe concentrations or amounts of pharmaceutical compositions according to the present invention which may be used to produce a favorable change in the disease or condition treated, whether that change is a reduction LDL-C levels, increase in HDL-C levels or other favorable physiological result.

As used herein, “combination therapy” or “therapeutic combination” means the administration of two or more therapeutic agents, such as therapeutic phospholipid composition and one or more lipid modifying agents, to prevent or treat a condition, for example a vascular condition, such as hyperlipidemia (as non-limiting examples, atherosclerosis, hypercholesterolemia or sitosterolemia), vascular inflammation, stroke, diabetes, obesity and/or reduce the level of sterol(s) (such as cholesterol) in the plasma. As used herein, “vascular” comprises cardiovascular, cerebrovascular and combinations thereof. The compositions, combinations and treatments of the present invention can be administered by any suitable means which produce contact of these compounds with the site of action in the body, for example in the plasma, liver or small intestine of a mammal or human. Such administration includes co-administration of these therapeutic agents in a substantially simultaneous manner, such as in a single tablet or capsule having a fixed ratio of active ingredients or in multiple, separate capsules for each therapeutic agent. Also, such administration includes use of each type of therapeutic agent in a sequential manner. In either case, the treatment using the combination therapy will provide beneficial effects in treating the condition. A potential advantage of the combination therapy disclosed herein may be a reduction in the required amount of an individual therapeutic compound or the overall total amount of therapeutic compounds that are effective in treating the condition. By using a combination of therapeutic agents, the side effects of the individual compounds can be reduced as compared to a monotherapy, which can improve patient compliance. Also, therapeutic agents can be selected to provide a broader range of complimentary effects or complimentary modes of action.

As used herein, “monotherapy” means when the active agent is used by itself and with other pharmaceutically acceptable excipients, carriers, binders, sweeteners, and the like. For example, when a statin composition is used in “monotherapy,” the composition contains only a statin and not therapeutic phospholipid compositions disclosed herein or any other active agents including other statins.

Health benefits derived from supplementation of the diet with omega-3 fatty acids, such as alpha-linolenic acid (“ALA”) (18:3), stearidonic acid (“STA”) (18:4), eicosatetraenoic acid (“ETrA”) (20:3), eicosatrienoic acid (“ETA”) (20:4), eicosapentaenoic acid (“EPA”) (20:5), docosapentaenoic acid (“DPA”) (22:5) and docosahexaenoic acid (“DHA”) (22:6), are well recognized and supported by numerous clinical studies and other published public and patent literature. Omega-3 fatty acids have been found to have beneficial effects on the risk factors for cardiovascular diseases, especially mild hypertension, hypertriglyceridemia and on coagulation factor VII phospholipid complex activity.

Omega-3 fatty acids, mainly eicosapentaenoic acid (EPA) and docosahexaenoic acid (DHA), have been shown to trigger the secretion of anti-inflammatory prostaglandins, thus providing the omega-3 fatty acid with anti-inflammatory properties. These effects directly inhibit the pro-inflammatory effects of omega-6 fatty acids, such as arachidonic acid. Consequently daily supplementation with EPA and DHA result in decreased formation of inflammatory mediators.

Omega-3 fatty acids are well known for their hypotriglyceridemic effects. Fish oil has been shown to decrease serum triglyceride concentrations from about 25% to about 40%, and decrease VLDL blood plasma levels. However, omega-3 fatty acids increase both LDL-C and HDL-C plasma levels. Harris, W. S. Clin. Cardiol. 1999, 22, (Suppl. II), II-4-II-43).

Omega-3 fatty acids can be obtained from marine organisms such as squid, fish, krill, etc. and are sold as dietary supplements. However, the uptake of omega-3 fatty acids by the body is not efficient and these raw oils contain other substances such as triglycerides and cholesterol which are known to cause deleterious side effects such as an increase in LDL-C. Certain fish oils have been developed as pharmaceutical-grade OM3-acid ethyl esters. One such OM3-acid ethyl ester is presently sold under the brand name Lovaza®. Studies have shown that Lovaza® can decrease plasma triglyceride levels in patients, however, Lovaza® has a negligible effect on raising good cholesterol (HDL-C). AMR101 is another ethyl ester form of OM3 fatty acids based on EPA with little or no DHA that is presently in clinical trials. AMR101 also appears to decrease triglycerides but also has a negligible effect on raising HDL-C.

U.S. Pat. No. 7,642,287, discloses a composition comprising omega-3 oil and pravastatin salts. This patent discloses the stability data of such compositions. The ability or extent of these compositions to lower LDL-C levels or to increase HDL-C levels is not disclosed.

Krill oil is extracted from Antarctic krill, Euphausia superba, a zooplantkton crustacean rich in phospholipids carrying long-chain omega-3 PUFAs, mainly EPA and EHA. Krill oil also contains potent antioxidants such as vitamins A and E, and the flavonoid astaxanthin. Additionally, Krill oil has a biomolecular profile of phospholipid conjugated omega-3 fatty acids which is significantly different than the usual profiles of fish oils. It is this phospholipid conjugation that facilitates the absorption of fatty acids through the intestinal wall, thus conferring better bioavailability and ultimately better long-lasting effects.

One such commercially available Krill oil is Neptune Krill Oil (NKO®). The source of NKO® is Antarctic krill (Euphasia superba). The composition of NKO® comprises the phospholipids phosphatidylcholine, phosphatidylethanolamine, phosphatidylinositol, and phosphatidylserine each carrying two omega-3 fatty acids (EPE:EPA, EPA:DHA, DHA:DHA), astaxanthin and flavanoids. No adverse events have been reported with NKO®.

U.S. Pat. No. 8,030,348 and U.S. Application Serial No. 13/189,714, the contents of which are incorporated herein in their entireties, disclose a Krill oil extract that comprises therapeutic phospholipid compositions. As disclosed, the therapeutic phospholipid compositions comprise compounds of Formula (I):

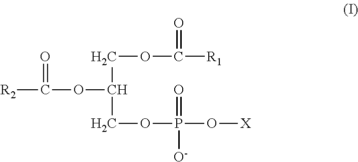

wherein R1 and R2, each together with the respective carboxyl groups to which each is attached, each independently represent a docosahexaenoic acid (DHA) or an eicosapentaenoic acid (EPA) residue, and X is —CH₂CH₂NH₃, —CH₂CH₂N(CH₃)₃, or

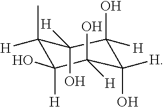

Additionally, all the embodiments of the therapeutic phospholipid compositions disclosed in U.S. Pat. No. 8,030,348 and U.S. Application Serial No. 13/189,714, are incorporated herein in their entireties. Furthermore, one skilled in the art can appreciate that any synthetic or natural variants of DHA or EPA can also be carried on the phospholipids of the present compositions.

Statins, although very effective in lowering cholesterol, have significant side effects, including but not limited to myositis, headache, rash, angioedema, gastrointestinal effects, myopathy, rhabdomyolysis and altered liver functions at their effective dosage levels.

Rosuvastatin (Crestor) is a more potent lipid-lowering agent than atorvastatin (Lipitor), pravastatin (Pravachol), simvastatin (Zocor), lovastatin (Mevacor) or fluvastatin (Lescol). Rosuvastatin has a similar benefit-risk profile as other statins. Garcia-Rodriguez et al. “The safety of rosuvastatin in comparison with other statins in over 25,000 statin users in the Saskatchewan Health Databases.” Pharmacoepidemiol. Drug. Saf. 2008, 17:953-961.

A recent meta-analysis of the therapeutic equivalence of statins concluded that a daily dose of atorvastatin 10 mg, fluvastatin 80 mg, lovastatin 40/80 mg, and simvastatin 20 mg could decrease LDL-C by 30%, to 42%. Wang et al. “A systematic review and meta analysis on the therapeutic equivalence of statins.” J. Clin. Pharm. Ther. 2010; 35: 139-151. In turn, fluvastatin 40 mg, lovastatin 10/20 mg, pravastatin 20/40 mg, and simvastatin 10 mg could decrease LDL-C by 20% to 30%. Reductions in LDL-C≧40% are necessary to achieve atherosclerosis regression. Grines C. L. “The role of statins in reversing atherosclerosis: what the latest regression studies show.” J. Interv. Cardiol. 2006, 19:3-9. The only two statins that could reduce LDL-C>40% were rosuvastatin and atorvastatin at a daily dose of 20 mg or higher. Based on these findings, a recent review concludes that in the absence of adverse events, rosuvastatin or atorvastatin at a dosage of ≧20 mg/d is probably the optimal statin and maintenance dosage for the vascular patient. Paraskevas et al. J. Vasc. Surg. 2011, 53(3), 837-844.

The statin-induced adverse effects may be dose related. Pedersen et al. “High dose atorvastatin vs. usual dose simvastatin for secondary prevention after myocardial infarction: the IDEAL study: a randomized controlled trial” JAMA 2005, 294: 2437-2445. A meta-analysis of prospective, randomized controlled trials evaluating intensive-and moderate-dose statin therapy for the reduction of cardiovascular events concluded that intensive therapy with atorvastatin or simvastatin 80 mg was associated with a significant increase in the risk for any adverse event, and adverse events requiring discontinuation of therapy. Silva et al. “Meta-analysis of drug-induced adverse events associated with intensive-dose stating therapy.” Clin. Ther. 2007, 29: 253-260. Intensive therapy was also associated with increased risk for abnormal liver function and elevated creatine kinase activity. Nevertheless, intensive statin therapy was also associated with reductions in cardiovascular death. Atorvastatin is associated with the greatest and fluvastatin with the lowest risk of adverse events. Simvastatin, pravastatin, and lovastatin have intermediate and similar risks which may cause adverse events. Silva et al. “Statin-related adverse events: a meta-analysis.” Clin. Ther. 2006, 28: 26-35.

The options for statin-intolerant patients include use of a different statin initiated at a lower dose with gradual up-titration; an alternate daily or weekly dosing of a statin with a long half life (e.g., rosuvastatin or atorvastatin); and combination of the lowest tolerated statin with a cholesterol absorption inhibitor (ezetimibe) and or bile acid sequestrant. Tziomlos et al. “Management of statin-intolerant high-risk patients.” Curr. Vasc. Pharmacol. 2010, 8: 632-637. However, whether these alternative options translate into a reduction of cardiovascular effects (as with high-dose daily statin therapy) remains to be proven.

It is desirable to seek alternatives to the current regime of statins for cardiac therapy due, inter alia, to their side effects, patient variability, intolerance, and cost. It is also highly desirable to seek therapeutic agents that exert synergistic effects when used in combination with lipid modifying agents, including statins. Such a combination therapy would decrease the effective dosage of statins, thereby reducing or completely eliminating the side effects.

Various clinical trials that included combination therapy of statin with ezetimibe, colesevelam, niacin, fenofibrate were shown to be effective in normalizing LDL-C. Dujovne et al. Curr. Atheroscler. Rep. 2011, 13(1), 12-22. The clinical trials with statins in combination with fish oil suggest that the most important effects are lowering serum triglyceride levels and beta-lipoproteins. These clinical trials also indicate that Omega-3 fatty acids, in most instances, have no effect on lowering LDL-C levels and may even raise them. Harris et al. “Effects of a low saturated fat, low cholesterol fish oil supplement in hypertriglyceridemic patients-a placebo controlled trial.” Ann. Intern. Med. 1988, 109 (6), 464-470.

Currently, there are no clear guidelines as to safe and effective dosage levels of statins when used in combination with other active agents. Though it is possible to lower the effective dosage of a statin in a combination therapy than the corresponding dosage in monotherapy, the safety and efficacy of such a dosage remain to be proven.

Serebruany et al. tested prescription omega 3 fatty acid ethyl esters (PO-3A) for outcome benefits in patients with coronary heart disease (CAD), arrythmias, and heart failure. The authors concluded that one week, short term therapy with PO-3A provided a modest reduction of platelet activity biomarkers, despite concomitant aspirin and statin therapy. Serebruany et al. Cardiology 2011, 118(3): 187-194.

A surprising and unexpected discovery, disclosed herein, is that co-administration of the therapeutic phospholipid compositions disclosed herein with lipid modifying agents, such as statins, increased the HDL-C levels and decreased LDL-C levels in subjects. It was also surprisingly discovered that administration of therapeutic phospholipid compositions alone also increased HDL-C levels and decreased LDL-C levels. Unexpectedly, it was found that when the therapeutic phospholipid composition is used in combination with lipid modifiying agents, such as statins, the resultant composition exerts a synergistic effect of lowering LDL-C levels and increasing HDL-C levels, even at very low dosages of statins.

In one aspect, the invention provides a method of treating a cardiometabolic disorder/metabolic syndrome, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid.

In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

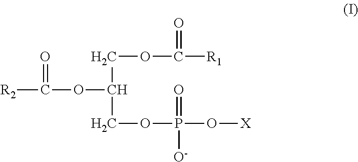

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

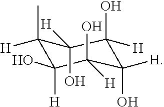

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In some embodiments, the compositions are capable of increasing HDL-C levels.

In another aspect, the invention provides a method of treating a cardiometabolic disorder/metabolic syndrome, the method comprising administering to a subject in need thereof a composition comprising an effective amount of a lipid modifying agent and a therapeutically effective amount of a phospholipid comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid comprises compounds of Formula (I):

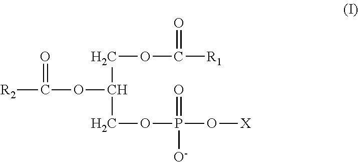

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

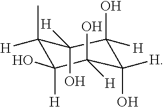

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In some embodiments, the composition is capable of increasing HDL-C levels.

In various embodiments, the cardiometabolic disorder/metabolic syndrome of any of the methods described herein is selected from atherosclerosis, dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoprotenemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

In yet another aspect, the invention provides a method of increasing HDL-C levels, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

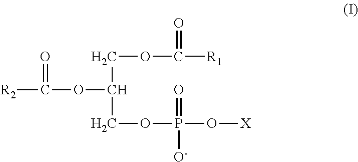

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

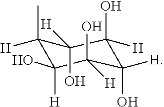

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount lipid modifying agent is less than the therapeutically effective amount used in monotherapy.

In still another aspect, the invention provides a method of increasing HDL-C levels and decreasing LDL-C levels, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-1′-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

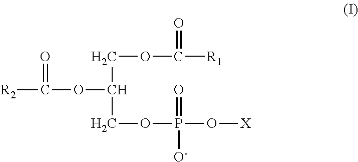

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

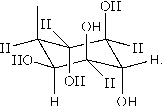

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In various embodiments, the HDL-C levels are increased by at least about 20% and LDL-C levels are decreased by at least about 10%.

In a further aspect, the invention provides a method of increasing HDL 2 levels' in a subject, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

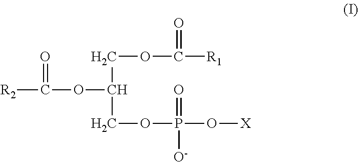

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

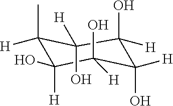

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In some embodiments, the composition is capable of increasing HDL-C levels.

In yet another aspect, the invention provides a method of decreasing triglyceride levels in a subject, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue, and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

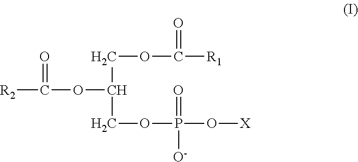

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

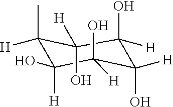

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In some embodiments, the composition is capable of increasing HDL-C levels.

In one aspect, the invention provides a method of increasing HDL-C levels and decreasing VLDL-C levels in a subject, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

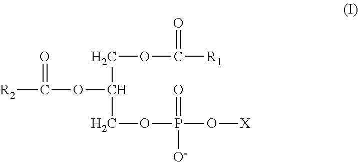

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

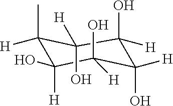

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In various embodiments, the HDL-C levels are increased by at least about 20% and VLDL-C levels are decreased by at least about 10%.

In yet a further aspect, the invention provides a method of reducing total cholesterol levels, the method comprising administering to a subject in need thereof an effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid. In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In another embodiment, the phospholipid composition comprises compounds of Formula (I):

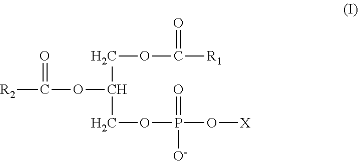

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

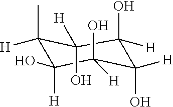

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic acid (20:4 (n-3)); EPA (20:5 (n-3)); behenic (22:0); erucic (22:1); docosadienoic (C22:2 (n-6)); adrenic (C22:4 (n-6)); ω-6 DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid. In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In a further embodiment, the fatty acid is selected from docosahexaenoic acid (DHA) or eicosapentaenoic acid (EPA).

In various embodiments, any of the above compositions may be pharmaceutical compositions.

In some embodiments, the therapeutically effective amount of lipid modifying agent is less than the therapeutically effective amount used in monotherapy.

In some embodiments, the composition is capable of increasing HDL-C levels.

In still a further aspect, the invention provides a method according to any of the above methods further comprising preventing, reducing or treating elevated cholesterol levels, atherosclerosis, hyperlipidemia, hypercholesterolemia, cardiovascular events and disease including coronary events and cerebrovascular events, and coronary artery disease and/or cerebrovascular disease, in a patient in need thereof.

In various embodiments, the above methods provide a statin that is selected from the group consisting of cerivastatin, atorvastatin, simvastatin, pravastatin, fluvastatin, lovastatin, rosuvastatin, and pitavastatin. In a specific embodiment, the statin is cerivastatin. In another specific embodiment, the statin is atorvastatin. In yet another specific embodiment, the statin is simvastatin. In a further specific embodiment, the statin is pravastatin. In still a further specific embodiment, the statin is fluvastatin.

In a specific embodiment, the statin is Lovastatin. In a specific embodiment, the statin is Rosuvastatin. In another specific embodiment, the statin is pitavastatin.

In various embodiments, the above methods provide a lipid modifying agent, including a statin, that is present in an effective dosage amount less than an effective dosage amount of what is used in monotherapy. In some embodiments, there is a lipid modifying agent, including a statin, that is present in less than about 50 mg/d. In some embodiments, there is a lipid modifying agent, including a statin, that is present in less than about 45 mg/d, or less than about 40 mg/d, or less than about 35 mg/d, or less than about 30 mg/d, or less than about 25 mg/d, or less than about 20 mg/d, or less than about 15 mg/d, or less than about 10 mg/d.

In various embodiments, the statin is present in an effective dosage amount less than an effective dosage amount of statin when used in monotherapy. In a specific embodiment, the statin is present in less than about 50 mg/d, or less than about 45 mg/d, or less than about 40 mg/d, or less than about 35 mg/d, or less than about 30 mg/d, or less than about 25 mg/d, or less than about 20 mg/d, or less than about 15 mg/d, or less than about 10 mg/d.

In one embodiment the therapeutically effective amount of a phospholipid composition is between about 0.5 g to at least about 1.0 g. In other embodiments, the therapeutically effective amount of a phospholipid composition is at least about 0.5 g, at least about 0.75 g, at least about 1.0 g, at least about 1.25 g, at least about 1.50 g, or at least about 3.0 g.

In some embodiments, the phospholipid composition can be administered before, simultaneously with, or after administration of the lipid modifying agent(s), such as, for example, statin(s).

In certain embodiments, the phospholipid composition and/or the lipid modifying agent are administered as a unit dosage formulation. The administration of the phospholipid composition and/or the statin can be by a route including, but not limited to of oral administration, nasal administration, rectal administration, intraperitoneal injection, intravascular injection, subcutaneous injection, transcutaneous administration, transdermal administration (by way of non-limiting example, patch sublingual), parenteral administration or intramuscular injection, and the like.

In certain embodiments, the subject is a mammal. In other embodiments, the mammal is diagnosed as having one or more symptoms of atherosclerosis. In certain embodiments, the mammal is a mammal diagnosed as at risk for stroke or atherosclerosis. In some embodiments, the mammal can be a human or a non-human mammal.

In certain other embodiments, the mammal is diagnosed as having one or more symptoms of hyperlipidemia. In some embodiments, the mammal can be a human or a non-human mammal.

In certain embodiments, the phospholipid compositions can be administered alone without any other active agents. In other embodiments, the phospholipid compositions can be co-administered with other active agents, for example, atorvastatin.

In one embodiment the co-administration is of rosuvastatin with phospholipid compositions.

In another embodiment the co-administration is of lovastatin with phospholipid compositions.

In another embodiment the co-administration is of fluvastatin with phospholipid compositions.

In yet another embodiment, the co-administration is of pravastatin with phospholipid compositions.

Another embodiment is co-administration of simvastatin with phospholipid compositions.

Another embodiment is co-administration of cerivastatin with phospholipid compositions.

In various embodiments, the co-administration proceeds, in order, phospholipid compositions followed by lipid modifying agent(s), such as statin(s).

In various embodiments, the co-administration proceeds, in order, lipid modifying agent(s), such as statin(s) followed by phospholipid compositions.

In various embodiments, the co-administration proceeds with simultaneous administration of lipid modifying agent(s), such as statin(s) and phospholipid compositions.

In addition to or in place of the lipid modifying agent(s), such as statin(s), the composition or medicaments of the present invention can comprise compounds possessing cholesterol absorption inhibitory activity, see for instance the compounds described in WO 93/02048, WO 94/17038, WO 95/08532, WO 95/26334, WO 95/35277, WO 96/16037, WO 96/19450, WO 97/16455, WO 02/50027, WO 02/50060, WO 02/50068, WO 02/50090, WO 02/66464, WO 04/000803, WO 04/000804, WO04/000805, WO 04/043457, and WO 04/081002; U.S. Pat. Nos. 5,756,470 and 5,767,115; U.S. Publication Nos. 2004/0180860 and U.S. 2004/0180861; and U.S. RE 37721, all of which are incorporated herein by reference in their entirety.

In an embodiment, this invention provides a method of mitigating one or more symptoms associated with atherosclerosis in a mammal. The method typically involves administering to the mammal an effective amount of one or more statins (e.g., cerivastatin, atorvastatin, simvastatin, pravastatin, fluvastatin, lovastatin. rosuvastatin, pitavastatin, etc.); and an effective amount of phospholipid compositions described herein, where the effective amount of the lipid modifying agent(s), such as statin(s) is lower than the effective amount of a statin administered without the phospholipid composition. In certain embodiments, the effective amount of the phospholipid composition is lower than the effective amount of the phospholipid composition administered without the lipid modifying agent(s), such as statin(s).

The phospholipid compositions can be administered before, simultaneously with, or after the lipid modifying agent(s), such as statin(s). The phospholipid compositions and/or the lipid modifying agent(s), such as statin(s) can be administered as a unit dosage formulation. The phospholipid compositions and/or lipid modifying agent(s), such as statin(s) can be administered by a route including, but not limited to oral administration, nasal administration, rectal administration, intraperitoneal injection, intravascular injection, subcutaneous injection, transcutaneous administration, inhalation administration, and intramuscular injection. In certain embodiments, the mammal is a human or non-human mammal diagnosed as having one or more symptoms of atherosclerosis and/or at risk for stroke and/or atherosclerosis. When the phospholipid compositions and lipid modifying agent(s), such as statin(s) are added together, they can be suitably formulated according to what is known by those skilled in the art and can be administered as a tablet, pill, syrup, spray, elixir or an injection. Any variation of the above dosage forms known to those skilled in the art (e.g. slow-release, rapid-release, sustained release, targeted release (intestines versus stomach), rapid dissolve microparticles, rapidly disintegrating microparticles, and mixtures of rapid dissolve and rapidly disintegrating microparticles etc.) can be employed.

### Modes of Administration

Administration of the therapeutic phospholipid compositions can be accomplished via any mode of administration for therapeutic agents. These modes include systemic or local administration such as, by way of non-limiting examples, oral, parenteral, transdermal, subcutaneous, or topical administration modes.

### Pharmaceutical Formulations

Depending on the intended mode of administration, the compositions can be in solid, semi-solid or liquid dosage form, such as, by way of non-limiting examples, injectables, tablets, pills, time-release capsules, elixirs, tinctures, emulsions, syrups, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, or any form that is known to those skilled in the pharmaceutical arts.

Illustrative pharmaceutical compositions are tablets and gelatin capsules comprising a therapeutic phospholipid composition neat, or if required, contains a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifiers; and/or g) an agent that enhances absorption of the compound such as cyclodextrin, hydroxypropyl-cyclodextrin, PEG400, and PEG200.

Other illustrative topical preparations include creams, ointments, lotions, aerosol sprays and gels, wherein the concentration of the therapeutic phospholipid composition ranges from about 0.1% to about 15%, w/w or w/v.

Liquid, particularly injectable, compositions can, by way of non-limiting example, be prepared by dissolution, dispersion, etc. For example, the therapeutic phospholipid composition is dissolved in or mixed with a pharmaceutically acceptable solvent such as, by way of non-limiting example, water, saline, aqueous dextrose, glycerol, ethanol, and the like, to thereby form an injectable isotonic solution or suspension. Proteins such as albumin, chylomicron particles, or serum proteins can be used to solubilize the therapeutic phospholipid composition.

### Dosing

The dosage regimen utilizing the therapeutic phospholipid compositions is selected in accordance with a variety of factors including type, species, age, weight, sex and medical condition of the subject; the severity of the condition to be treated; the route of administration; the renal or hepatic function of the subject; and the particular therapeutic phospholipid composition employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 10000 mg of the therapeutic phospholipid composition per day. Dosages for in vivo or in vitro use can contain about 20, 50, 75, 100, 150, 250, 500, 750, 1000, 1250, 2500, 3500, 5000, 7500 or 10000 mg of the therapeutic phospholipid composition. Effective blood plasma levels after administration of the therapeutic phospholipid composition to a subject can range from about 0.002 mg to about 100 mg per kg of body weight per day. Appropriate dosages of the therapeutic phospholipid composition can be determined as set forth in L. S. Goodman, et al., The Pharmacological Basis of Therapeutics, 201-26 (5th ed. 1975).

The therapeutic phospholipid compositions can be administered in a single daily dose, or the total daily dosage can be administered in divided doses of two, three or four times daily. To be administered in the form of a transdermal delivery system, the dosage administration can be continuous rather than intermittent throughout the dosage regimen. In some embodiments, the therapeutic phospholipid composition and the therapeutic agent can be administered simultaneously. In other embodiments, the therapeutic phospholipid composition and the therapeutic agent can be administered sequentially. In still other embodiments, the therapeutic phospholipid composition can be administered daily and the therapeutic agent can be administered less than daily. In still other embodiments, the therapeutic phospholipid composition can be administered daily and the therapeutic agent can be administered more than once daily.

Pharmaceutical dosage forms of the invention can be administered orally, parenterally, by inhalation, spray, topically, rectally, nasally, buccally, vaginally or via an implanted reservoir. In some embodiments, oral and parenteral pharmaceutical compositions and dosage forms are used. In some embodiments, the oral dosage form is a homogeneous or heterogeneous formulation, a parenteral dosage form, or a capsule formulation (including without limitation hard gelatin capsules, starch capsules, HPMC capsules, and soft elastic gelatin capsules). In other embodiments, dosage forms include an intradermal dosage form, an intramuscular dosage form, a subcutaneous dosage form, and an intravenous dosage form.

Pharmaceutical unit dosage forms of this invention are suitable for oral, mucosal (e.g., nasal, sublingual, vaginal, buccal, or rectal), parenteral (e.g., intramuscular, subcutaneous, intravenous, intraarterial, or bolus injection), topical, or transdermal administration to a patient. Examples of dosage forms include, but are not limited to: capsules, dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., non-aqueous liquid suspensions, solutions, and elixirs, and liquid dosage forms suitable for parenteral administration to a patient.

The composition, shape, and type of dosage forms of the invention will typically vary depending on their use. For example, a dosage form used in the acute treatment of a disease or disorder may contain larger amounts of the active ingredient than a dosage form used in the chronic treatment of the same disease or disorder. Similarly, a parenteral dosage form may contain smaller amounts of the active ingredient than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990) or Remington: The Science and Practice of Pharmacy, 19th ed., Mack Publishing, Easton Pa. (1995).

In another embodiment, a pharmaceutical composition or medicament of the present invention comprises about 500, 600, 700, 800, 900, 1000, 1100, 1200, 1300, 1400, 1500, 2000, 2500, 3000, 3500, or 5000 mg of therapeutic phospholipid composition and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a lipid modifying agents(s).

In another embodiment, a pharmaceutical composition or medicament of the present invention comprises about 500, 600, 700, 800, 900, 1000, 1100, 1200, 1300, 1400, 1500, 2000, 2500, 3000, 3500, or 5000 mg of therapeutic phospholipid composition and about 1, 2, 3, 4, 5, 10, 15, 20, 25, 30, 35, 40, 45, 50, 55, 60, 65, 70, 75, 80, 90, 100, 110, 120, 130, 140, 150, or 160 mg of a statin(s).

In an embodiment, a pharmaceutical composition or medicament of the present invention comprises about 500, 600, 700, 800, 900, 1000, 1100, 1200, 1300, 1400, 1500, 2000, 2500, 3000, 3500, or 5000 mg of therapeutic phospholipid composition and about 1, 2, 3, 4, 5, 10, 15, 20, 25, 30, 35, 40, 45, 50, 55, 60, 65, 70, 75, or 80 mg of atorvastatin.

### Oral Dosage Forms

Pharmaceutical compositions of the invention that are suitable for oral administration can be presented as discrete dosage forms, such as, but not limited to, capsules as described above and liquids, such as but not limited to, syrups, elixirs, solutions or suspensions. Such dosage forms may be prepared by methods of pharmacy well known to those skilled in the art. See generally, Remington's Pharmaceutical Sciences, 18th ed., Mack Publishing, Easton Pa. (1990) or Remington: The Science and Practice of Pharmacy, 19th ed., Mack Publishing, Easton Pa. (1995).

### Controlled-Release Dosage Forms

Statins can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, “Controlled Release Dosage Form Design.” 2 Technomic Publishing, Lancaster, Pa.: 2000.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with compositions of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185; each of which is incorporated herein by reference in its entirety. These dosage forms can be used to provide slow or controlled-release of one or more active ingredients using, for example, hydroxypropylmethyl cellulose, other polymer matrices, gels, penneable membranes, osmotic systems (such as OROS®, Alza Corporation, Mountain View, Calif. USA), multilayer coatings, microparticles, liposomes, or microspheres or a combination thereof to provide the desired release profile in varying proportions.

The present invention can also be used in an extended-release form as disclosed in U.S. application Ser. No. 10/635,221 which is incorporated herein in its entirety. This method involves a bolus injection of the drug which implants the drug intramuscularly to form an intramuscular depot. Especially, with respect to the current invention, the statins can be administered this way, and the therapeutic phospholipid composition can then be administered sequentially (before or after the injection). Alternatively, statins and the therapeutic phospholipid composition can be administered in one bolus injection. The statins can be derivatized according to knowledge in the art to suit such an intramuscular deposition.

In one aspect, there is provided a composition comprising a therapeutically effective amount of a lipid modifying agent and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid.

In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid.

In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In still another aspect, there is provided a composition comprising a therapeutically effective amount of a lipid modifying agent and a therapeutically effective amount of a phospholipid composition comprising compounds of Formula (I):

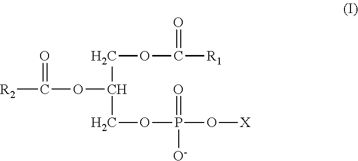

wherein one of R1 and R2, each together with the respective carboxyl groups to which each is attached is selected from a docosahexaenoic acid (DHA) or an eicosapentaenoic acid (EPA) residue, and the other one of R1 and R2 is a fatty acid, and X is —CH₂CH₂NH₃, —CH₂CH₂N(CH₃)₃, or

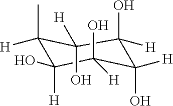

In one embodiment, the lipid modifying agent is selected from the group consisting of statins, ezetimibe, fibrates, niacinamide, and niacin.

In one embodiment, the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

In another embodiment, the fatty acid is an omega 3-fatty acid or a fatty acid that is metabolized to an omega 3-fatty acid.

In some embodiments, the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

In some embodiments the fatty acid is docosahexaenoic acid (DHA) or an eicosapentaenoic acid (EPA).

In other embodiments, the composition is a pharmaceutical composition.

In still other embodiments, the therapeutically effective amount of lipid modifying agent is less than the therapeutically effective amount used in monotherapy.

In various embodiments, the compounds are in a concentration of between about 10% (w/w (phospholipids/total composition)) up to 50% (w/w (phospholipids/total composition)). In other various embodiments, the compounds are in a concentration of between about 15% (w/w (phospholipids/total composition)) up to 45% (w/w (phospholipids/total composition)). In various embodiments, the compounds are in a concentration of between about 20% (w/w (phospholipids/total composition)) up to 40% (w/w (phospholipids/total composition)). In still other various embodiments, the compounds are in a concentration of about 25% (w/w (phospholipids/total composition)), about 30% (w/w (phospholipids/total composition)), about 32% (w/w (phospholipids/total composition)), about 33% (w/w (phospholipids/total composition)), about 35% (w/w (phospholipids/total composition)), or about 37% (w/w (phospholipids/total composition)).

In various embodiments, the compounds include therapeutic phospholipid composition which comprises about the same amount of EPA and DI-TA. In some embodiments, the therapeutic phospholipid composition comprises more DHA than EPA. In some embodiments, the therapeutic phospholipid composition comprises more EPA than DHA. In other embodiments, the DHA and EPA comprise at least about 32% w/w of the total lipids in the composition or at least about 35% w/w of the total lipids in the composition.

In various embodiments, the compounds further comprise an additional lipid. In some embodiments the additional lipid is selected from the group consisting of a monoglycerides, triglycerides, cholesterols and mixtures thereof. In some embodiments, the compositions further comprise about 4% w/w of free fatty acids or about 5% w/w of free fatty acids. In various embodiments, the compositions further comprise polyunsaturated fatty acids. In some embodiments the polyunsaturated fatty acids comprise at least 15% w/w of the total lipids in the composition, at least 40% w/w of the total lipids in the composition, at least 45% w/w of the total lipids in the composition. In various embodiments the polyunsaturated fatty acids are omega-3 fatty acids.

In some embodiments the compositions further comprises a metal. In various embodiments, the metal is zinc, selenium or a mixture thereof. In specific embodiments, the zinc comprises at least 0.005 mg/100 g of the composition and the selenium comprises less than 3 mg/100 g of the composition.

In a specific embodiment, the fatty acid composition of the lipids in the composition is about:

| | Total | Phospha- | Phosphati- |
|---|---|---|---|
| | Phospholipid | tidylcholine | dylethanolamine |
| Fatty Acids | Fatty Acid % | Fatty Acid % | Fatty Acid % |
| C14:0 MYRISTIC | 2 | 2 | 0.7 |
| C14:1 MYRISTOLEIC | 1 | | |
| C15:0 | 0.2 | 0.3 | 0.3 |
| PENTADECANOIC | | | |
| C16:0 PALMITIC | 24 | 27 | 24 |
| C16:1 PALMITOLEIC | 2 | 2 | 0.7 |
| C18:0 STEARIC | 1 | 1 | 3 |
| C18:1 OLEIC | 9 | 12 | 24 |
| C18:2n6 LINOLEIC | 2 | 2 | 0.8 |
| C18:3n6 GLA | 1 | 0.3 | |
| C18:3n3 ALA | 1 | 1 | |
| C18:4n3 OTA | 2 | 2 | 0.3 |
| C20:0 ARACHIDIC | | | |
| C20:1 cis-11- | 0.5 | 0.6 | 0.7 |
| EICOSENOIC | | | |
| C20:2n6 | | | |
| EICOSADIENOIC | | | |
| C20:3n6 METHYL | | 0.2 | |
| ETA | | | |
| C20:4n6 | 0.6 | 0.7 | 0.6 |
| ARACHIDONIC | | | |
| C20:3n3 Homo-γ- | | | |
| LINOLENIC | | | |
| C20:4n3 | | | |
| C20:5n3 EPA | 27 | 32 | 13 |
| C22:0 BEHENIC | | | |
| C22:1 ERUCIC | 1 | 1.5 | |
| C22:2n6 | | | |
| C22:4n6 | | | |
| C22:5n6 METHYL | | | |
| DPA | | | |
| C22:5n3 DPA | | 1.0 | |
| C22:6n3 DHA | 25 | 14 | 32 |
| C24:0 LIGNOCERIC | | | |
| C24:1 NERVONIC | | | |
| Total | 100.0 | 100 | 100 |

In some embodiments, the total fatty acid composition of all the lipids in the composition is about:

| Sample Fatty Acid Composition | % |
|---|---|
| C14:0 | ≧3.00 |
| C14:1 | ≧0.01 |
| C15:0 | ≧0.3 |
| C16:0 | ≧20.00 |
| C16:1 | ≧3.25 |
| C18:0 | ≧1.00 |
| C18:1 | ≧10.00 |
| C18:2n6 | ≧2.00 |
| C18:3n6 GLA | ≧0.04 |
| C18:3n3 ALA | ≧0.01 |
| C18:4n3 | ≧1.50 |
| C20:0 | ≧0.05 |
| C20:1 | ≧1.00 |
| C20:2n6 | ≧0.05 |
| C20:3n6 | ≧0.05 |
| C20:4n6 | ≦0.50 |
| C20:3n3 | ≧0.01 |
| C20:4n3 | ≧0.20 |
| C20:5n3 EPA | ≧25.00 |
| C22:0 | ≧0.01 |
| C22:1 | ≧1.50 |
| C22:2n6 | ≧0.03 |
| C22:4n6 | ≧0.01 |
| C22:5n6 | ≧0.01 |
| C22:5n3 DPA | ≧0.50 |
| C22:6n3 DHA | ≧10.00 |
| C24:0 | ≧0.01 |
| C24:1 | ≧0.05 |

In other embodiments, the total fatty acid composition of all the lipids is about:

| | |
|---|---|
| Saturated (g/100 g lipid) | ≧22.00 |
| Monounsaturated (g/100 g lipid) | ≧11.00 |
| Polyunsaturated (g/100 g lipid) | ≧35.00 |
| Omega-3 (g/100 g lipid) | ≧30.00 |
| Omega-6 (g/100 g lipid) | ≧1.00 |

In yet another embodiment, the compositions, comprise about:

| | |
|---|---|
| Monoglycerides (MG) (g/100 g sample) | ≧0.7 |
| Triglycerides (TG) (g/100 g sample) | ≧3.00 |
| Free Fatty Acids (FFA) (g/100 g sample) | ≧5.00 |
| Cholesterol (g/100 g sample) | ≦2.00 |
| Total Phospholipids (PL) (g/100 g sample) | ≧40.00 |
| Phosphatidyl Ethanolamine (PE) (g/100 g sample) | ≧2.50 |
| Phosphatidyl Inositol (PI) (g/100 g sample) | ≧0.20 |
| Phosphatidyl Serine (PS) (g/100 g sample) | ≧0.20 |
| Phosphatidyl Choline (PC) (g/100 g sample) | ≧35.00 |
| Sphingomyelin (g/100 g sample) | ≧0.50 |
| Vitamin A (μg/100 g ml) | ≧1,400 |
| Vitamin E (μg/100 g sample) | ≧15 |
| Beta-Carotene (μg/100 g ml) | ≧1,600 |
| [[≧]]Astaxanthin (g/100 g ml) | ≧10 |
| Canthaxanthin (mg/100 g ml) | ≧10 |
| Flavonoid (mg/100 ml) | ≧7.0 |

In an embodiment, the composition further comprises an antioxidant. In a specific embodiment, the antioxidant is selected from the group consisting of vitamin A, vitamin E, carotenoid, beta-carotene, astaxanthin, canthaxanthin, flavonoids and mixtures thereof.

In some embodiments, the compositions further comprise about 0% w/w of free fatty acids.

In various embodiments, the compositions comprise a statin that is selected from the group consisting of cerivastatin, atorvastatin, simvastatin, pravastatin, fluvastatin, lovastatin, rosuvastatin, and pitavastatin. In a specific embodiment, the statin is cerivastatin; or Atorvastatin, or simvastatin, or pravastatin, or fluvastatin, or Lovastatin, or Rosuvastatin, or pitavastatin.

The present invention also encompasses kits. For instance, in one aspect, the invention provides a kit comprising two tablets or capsules in which a first tablet or capsule comprises a lipid modifying agent and a second tablet or capsule comprises a phospholipid composition.

In one embodiment, the kit further comprises a blister package.

This invention is further illustrated by the following examples that should not be construed as limiting. Those skilled in the art should, in light of the present disclosure, appreciate that many changes can be made to the specific embodiments which are disclosed and still obtain a like or similar result without departing from the spirit or scope of the invention.

### EXAMPLES

### Methods

In all examples, randomized, placebo controlled, multi-center, double blind studies were undertaken. Subjects were patients with hyperlipidemia who can maintain a healthy diet. Patients were given pharmaceuticals as specified in particular Examples. To be eligible for the study, patients must have been: male or female and between 18-85 years of age, have a total cholesterol level of between 5-9.0 mmol/l, and have given written informed consent. Potential subjects were excluded if they presented with familial hypercholesterolemia or severe high cholesterol levels of greater than 9.0 mmol/l. Further, patients were excluded if a lipid lowering treatment was initiated less than six months prior to enrolment, if the lipid lowering treatment being used was anything other than statin, and if the daily dose of statin was higher than 10 mg.

Subjects were assessed at the beginning and end of the study for certain blood parameters, including after the three months treatment period for cholesterol; triglycerides; LDL cholesterol; HDL cholesterol; cholesterol/HDL ratio; and glucose.

### Example 1

### Compounds of Formula I Cause a Larger Decrease in LDL and Increase in HDL Levels than Fish Oil

A double blind prospective randomized clinical study was undertaken in patients with hypercholerolemia (n=60). One subset of patients received either 1.0-1.5 g/daily NKO® (i.e. 1 g/day for subjects with a BMI≦30 or 1.5 g/day for subjects with a BMI>30) or 3.0 g/daily fish oil (180 mg EPA/120 mg DHA) for 3 months. Percent changes in levels of cholesterol, triglycerides, LDL, HDL and glucose were examined. Placebo control groups were also examined.

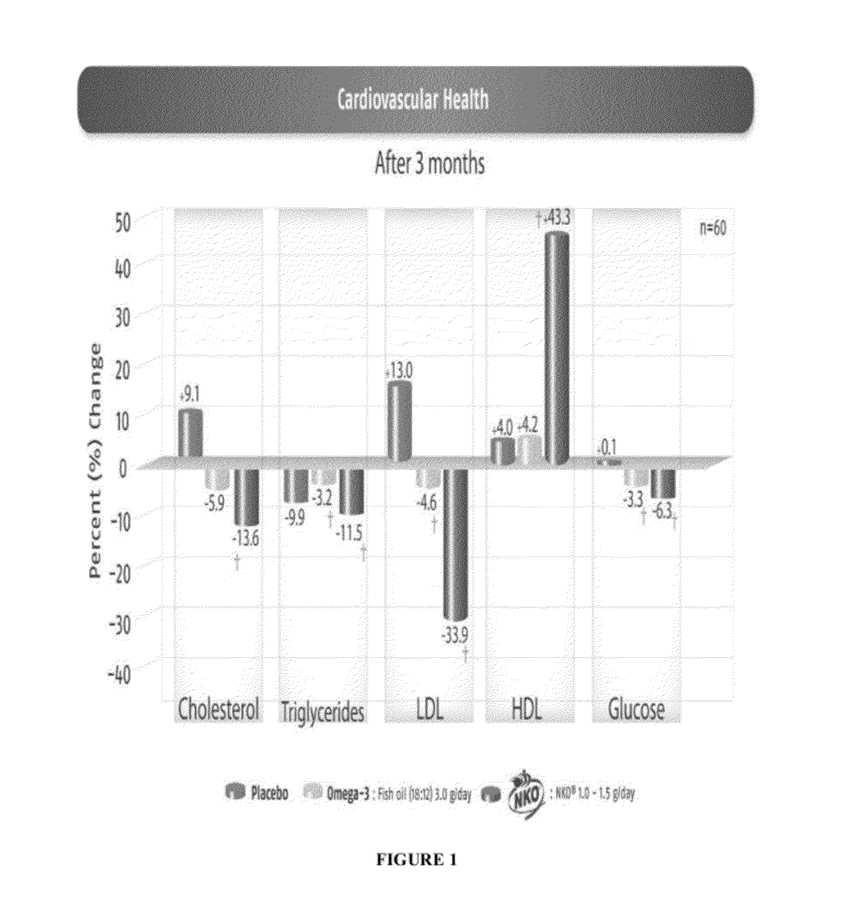

FIG. 1 shows the effects of placebo (left histogram of each subset), fish oil (middle histogram of each subset) and NKO® (right histogram of each subset) for a variety of clinical subsets: cholesterol, triglycerides, LDL, HDL and glucose. As shown in FIG. 1, NKO® treated patients showed a dramatic reduction in LDL levels: a −33.9% change. Fish oil patients showed only a −4.6% change. Therefore the NKO® effect was over seven times greater. Further, NKO® caused a significant increase in HDL levels: causing a 43.3% increase. This was over ten-fold greater than the effect observed with fish oil (4.2% increase).

### Example 2

### The Effect of Statins on LDL and HDL Levels is Improved by Co-Administration with the Compounds of Formula I

A subset of a double blind prospective randomized clinical study in patients diagnosed with hyperlipidemia (n=44) who were on a 10 mg dose of statin regimen for at least 6 months, received either 10 g/daily statins or a combination of 10 g/daily statins and 1.0-1.5 g/daily of NKO® (i.e. 1 g/day for subjects with a BMI≦30 or 1.5 g/day for subjects with a BMI>30) was examined.

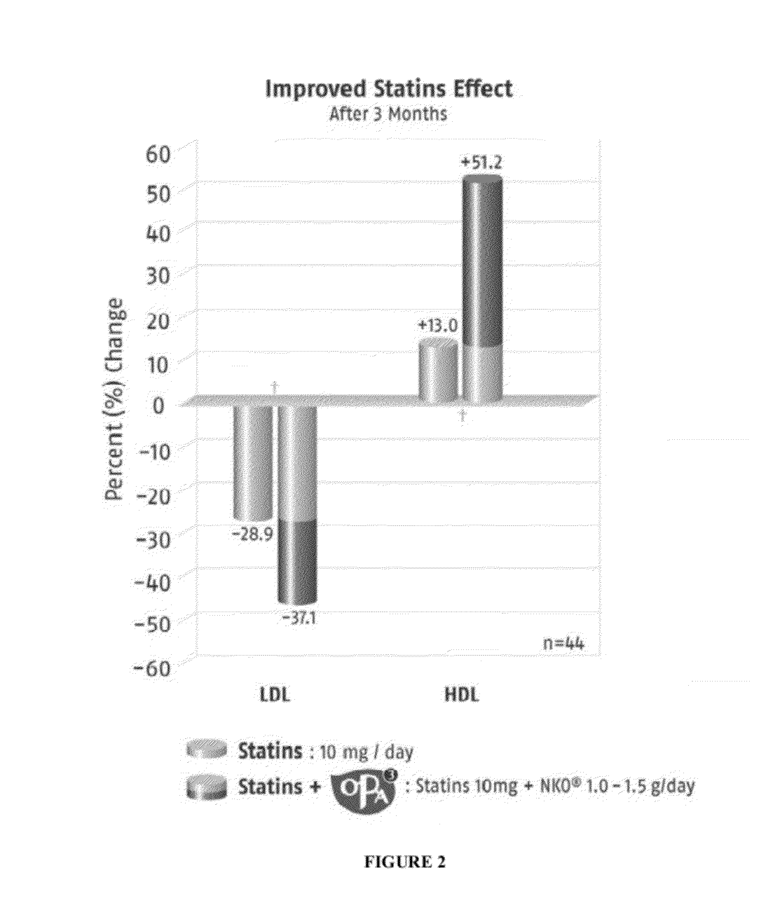

FIG. 2 shows the results of this study with the statin-only histogram on the left (one shade) and the combination therapy histogram on the right (two shades). The combination therapy showed a superior effect in LDL reduction and HDL increase. As to the former, the combination therapy caused a −37.1% change in LDL, compared to −28.9% in statins alone: a difference of −8.2%. As to the latter, the combination therapy caused a +51.2% change in LDL, compared to +13.0% in statins alone: a difference of 38.2%, which is an almost four-fold effect.

### Example 3

### The Combination of Compounds of Formula I and Statins Shows Superior Effects on LDL and HDL Levels as Compared to Fish Oil Combinations

The present inventors have determined that the combination therapy of compounds of Formula I and statins is superior to alternative combination therapies, such as fish oil and statins.

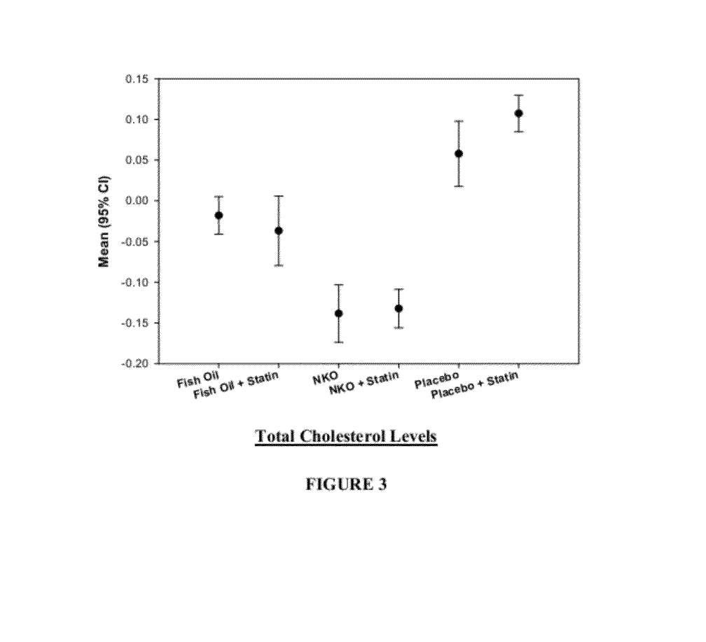

FIG. 3 shows the total cholesterol levels (absolute change) in a double blind prospective randomized clinical study in patients diagnosed with hyperlipidemia that were on a 10 mg dose of statin regimen for at least 6 months. Patients received one of six possible treatments: fish oil (3.0 g/daily fish oil (180 mg EPA/120 mg DHA)), fish oil and statins (10 mg/daily), NKO® (1.0-1.5 g/daily; i.e. 1 g/day for subjects with a BMI≦30 or 1.5 g/day for subjects with a BMI>30), NKO® and statins (10 mg/daily), placebo (3 g/daily), or placebo and statins (10 mg/daily).

As FIG. 3 shows, the NKO® and NKO® and statins combination groups had a noticeable decrease in total cholesterol levels when compared to the fish oil and placebo groups. To further, tease out the effects of the NKO® and statins combination, the inventors assessed the LDL and HDL levels in patients.

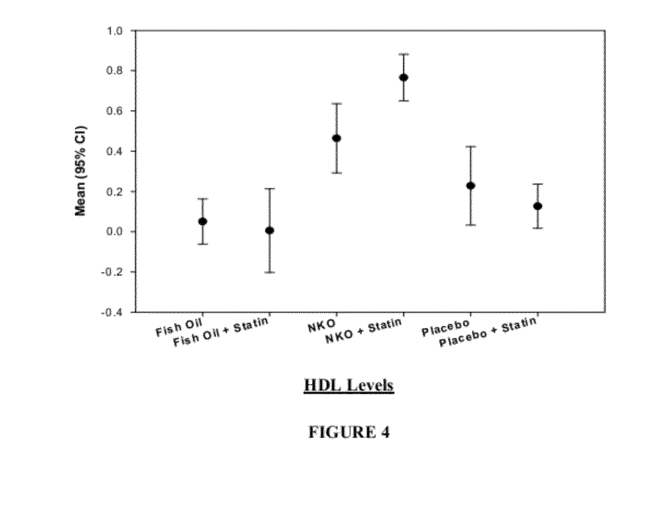

FIG. 4 shows HDL levels (absolute change) in a double blind prospective randomized clinical study in patients diagnosed with hyperlipidemia who were on a 10 mg dose of statin regimen for at least 6 months. Patients received one of six possible treatments: fish oil (3.0 g/daily fish oil (180 mg EPA/120 mg DHA)), fish oil and statins (10 mg/daily), NKO® (1.0-1.5 g/daily; i.e. 1 g/day for subjects with a BMI≦30 or 1.5 g/day for subjects with a BMI>30), NKO® and statins (10 mg/daily), placebo (3 g/daily), or placebo and statins (10 mg/daily).

As FIG. 4 shows, the NKO® and NKO® and statins groups had a noticeable increase in HDL levels when compared to the fish oil and placebo groups. First, both of the NKO® groups (alone and in combination) had superior clinical effects as compared to the fish oil and placebo groups. Further, the NKO® and statins group displayed an increased amount of HDL as compared to the NKO® alone group with the former showing a mean value of around 0.8 and the latter showing a mean value of around 0.45.

Further still, the effects of statins are demonstrated with the placebo and statins combination therapy group. Importantly, statins did not increase the amount of HDL levels noticeably. The statin effect was eight-fold less that the NKO® and statins group, further demonstrating the superiority of the inventive combination.

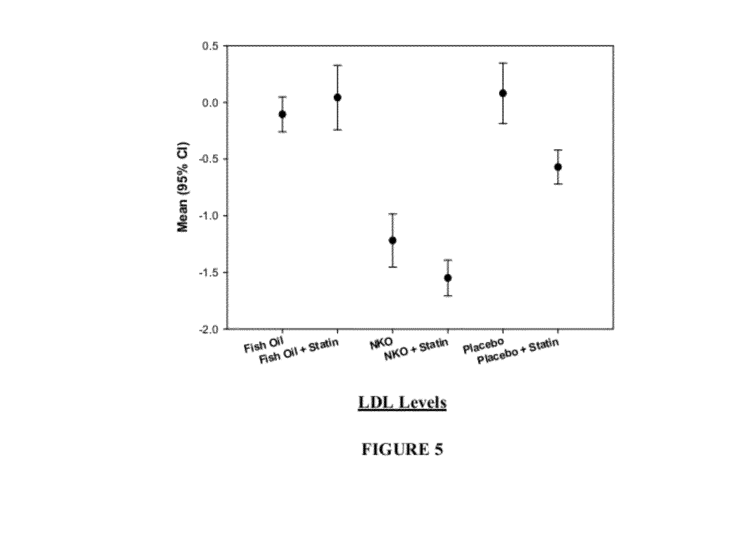

FIG. 5 shows LDL levels (absolute change) in a double blind prospective randomized clinical study in patients diagnosed with hyperlipidemia who were on a 10 mg dose of statin regimen for at least 6 months. Patients received one of six possible treatments: fish oil (3.0 g/daily fish oil (180 mg EPA/120 mg DHA)), fish oil and statins (10 mg/daily), NKO® (1.0-1.5 g/daily; i.e. 1 g/day for subjects with a BMI≦30 or 1.5 g/day for subjects with a BMI>30), NKO® and statins (10 mg/daily), placebo (3 g/daily), or placebo and statins (10 mg/daily).

As FIG. 5 shows, the NKO® and NKO® and statins groups had a noticeable decrease in LDL when compared to the fish oil and placebo groups. For instance, neither of the fish oil groups caused a reduction that was lower than about −0.25. On the other hand the NKO® and statins group showed a reduction of about −1.6.

Further still, the effects of statins are demonstrated with the placebo and statins combination therapy group. Importantly, statins did not decrease the amount of LDL levels to levels comparable with the inventive combination therapies. The statin effect was about three-fold less that the NKO® and statins group, further demonstrating the superiority of the inventive combination.

### Example 4

### The Combination of Compounds of Formula I and Statins Shows Superior Effects on Fatal Cardiovascular Heart Event Risk as Compared to Fish Oil Combinations

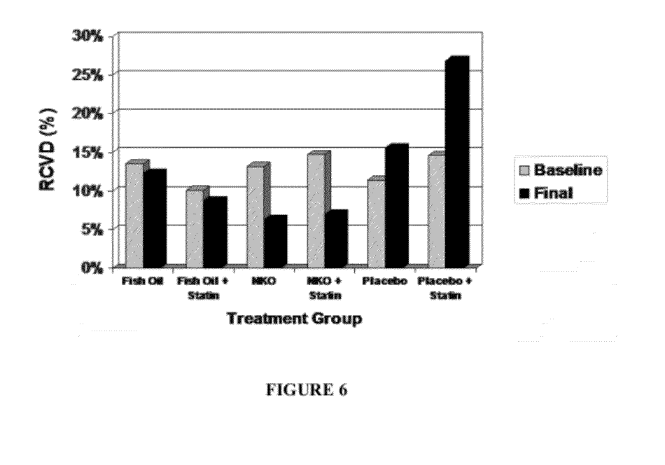

As shown in FIG. 6, the data was analyzed under the Framingham risk model. Fatal cardiovascular event absolute risk was analyzed in a double blind prospective randomized clinical study in patients diagnosed with hyperlipidemia who were on a 10 mg dose of statin regimen for at least 6 months, who received either 1.0-1.5 g/daily of NKO® (i.e., 1 g/day for subjects with a BMI≦30 or 1.5 g/day for subjects with a BMI>30) or 3.0 g/daily fish oil (180 mg EPA/120 mg DHA). Framingham Risk Score data analysis showed that patients treated with NKO® or the combination of NKO® and statins produced a sizable reduction in the Framingham Risk Score (R_{CVD}) comparing baseline to final (i.e., before and after treatment) which was considerably more dramatic than in the fish oil and fish oil and statins combination groups. Placebo controls showed an increase in Framingham Risk Score.

### Example 5

### The Combination of Compounds of Formula I and Statins Shows Superior Effects when Analyzed Across Subject Subgroups

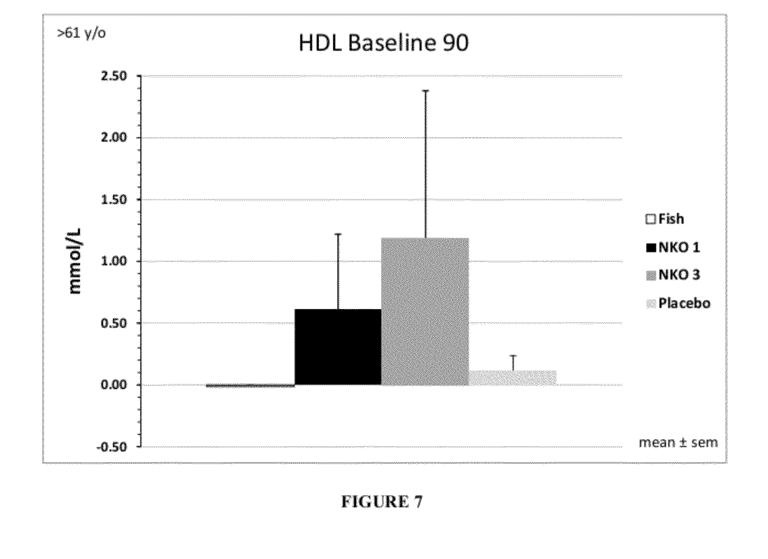

The inventors analyzed the effects of the combination therapy across subject subgroups: subjects over the age of 61 years old; subjects with initial/pre-treatment LDL levels of 2.5-3.5 mmol/L or greater than 3.5 mmol/L; and subjects with high, moderate, and low initial/pre-treatment HDL levels. As shown in FIGS. 7-9, the HDL increasing and LDL lowering effect was maintained in all of the subgroups.

FIG. 7 shows the effect of the combination therapy of 10 mg/day dose of statin and either 1.0 g/daily or 3 g/daily of NKO® (second and third from left bars), 10 mg/day dose of statin and 3.0 g/daily fish oil (left bar), or 10 mg/day dose of statin and placebo (right bar) on HDL levels in subjects over 61 years old. This data shows that the combination therapy caused a significant increase in HDL levels in the over 61 years old subgroup as the reported values are those presented by patients after 90 days of treatment. This effect was considerably larger than that observed for the fish oil and placebo groups (in combination with statins). The larger dose of NKO (3 g/daily) produced a larger effect than the smaller dose of NKO (1 g/daily), both in combination with statins.

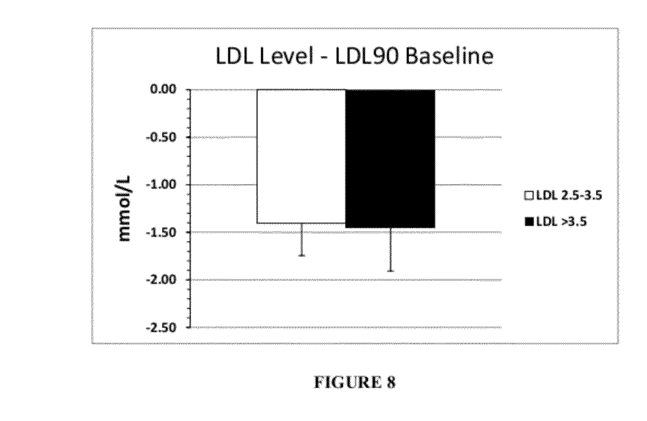

FIG. 8 shows the LDL reducing effect of the combination therapy (10 mg dose of statin and 1.0-1.5 g/daily NKO®) on levels of LDL in subjects after 90 days of treatment. Subjects in groups with both pre-treatment levels of LDL (i.e. 2.5-3.5 mmol/L or >3.5 mmol/L) showed a reduction in LDL levels. This data shows that the combination therapy achieves a significant reduction in LDL levels at both baseline levels of LDL.

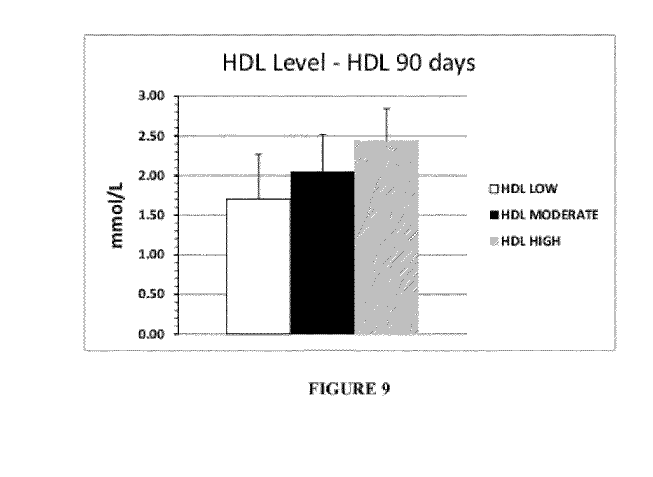

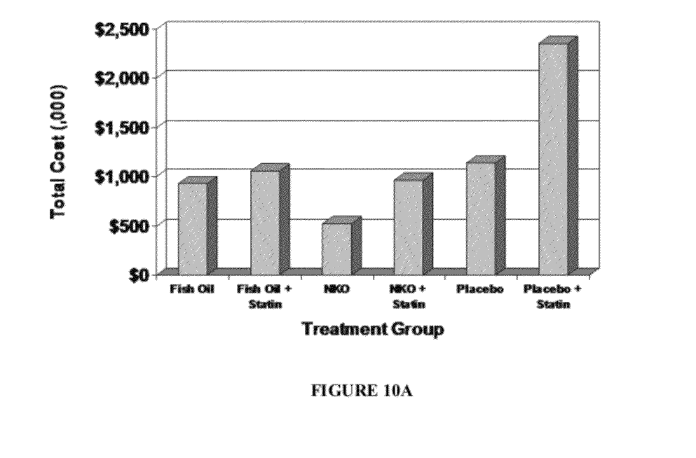

FIG. 9 shows the HDL increasing effect of the combination therapy (10 mg dose of statin and 1.0-1.5 g/daily NKO®) on levels of HDL in subjects after 90 days of treatment. Subjects in groups with all pre-treatment levels of HDL (i.e. low (less than 40 mg/dL), moderate (between 40-60 mg/dL), and high (greater than 60 mg/dL) showed an increase in HDL levels. This data shows that the combination therapy achieves a significant increase in HDL levels at all baseline levels of HDL.

### Example 6

### The Combination Treatment Displays Superior Cost Effectiveness

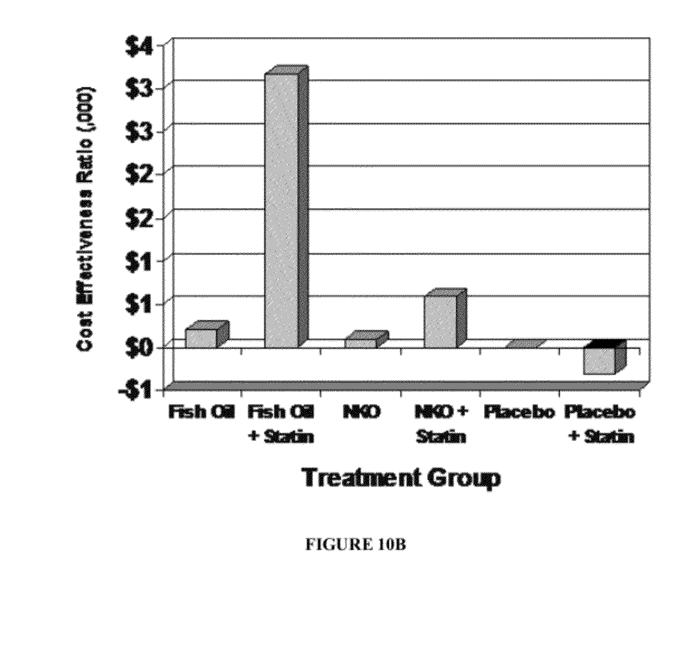

The combination treatment of NKO® and statins proved to be more cost effective than other treatment regimes, except NKO® alone, considering the added cost of treatment for cardiovascular events and side effects. As shown in FIG. 10A, after a 90 day treatment, the lowest overall cost was observed for the patients treated with NKO® and NKO® and statins co-administration. The overall cost of NKO® Administration was nearly $525,000 while the cost of the NKO® and statins combination was around $965,000. These costs were far less than the costs associated with the other analyzed combinations: fish oil and statins (˜$1.1 mill.) and placebo and statins (more than $2.1 mill.).

Not only was cost minimized, but also the treatment described herein was among the most cost effective. As shown in FIG. 10B, after a 90 day treatment, the lowest overall cost for every event prevented was observed for the patients treated with NKO® and NKO® and statins combination. For example, the cost effectiveness ratio of NKO® and statins combination was over three times lower than the fish oil and statins combination.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed is:

1. A method of treating a cardiometabolic disorder/metabolic syndrome, the method comprising administering a therapeutically effective amount of a lipid modifying agent and a composition comprising therapeutically effective amount of a phospholipid comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid, to a subject in need thereof.

2. The method of claim 1, wherein the lipid modifying agent is selected from the group consisting of statins, ezetimibe, fibrates, niacinamide, and niacin.

3. The method of claim 1, wherein the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

4. The method of claim 1, wherein the fatty acid is an omega 3-fatty acid or a fatty acid that is metabolized to an omega 3-fatty acid.

5. The method of claim 4, wherein the omega 3-fatty acid or a fatty acid that is metabolized to an omega 3-fatty acid is selected from the group consisting of hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

6. The method of claim 1, wherein the phospholipid composition is a compound of Formula (I): 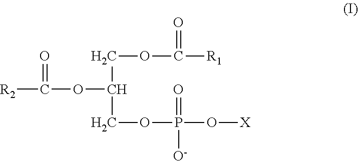
wherein R1 and R2, each together with the respective carboxyl groups to which each is attached, each independently represent a docosahexaenoic acid (DHA) or an eicosapentaenoic acid (EPA) residue, and X is —CH₂CH₂NH₃, —CH₂CH₂N(CH₃)₃, or

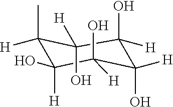

7. The method of claim 1, wherein the cardiometabolic disorder/metabolic syndrome is selected from atherosclerosis, dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoprotenemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

8. The method of claim 1, further comprising preventing, reducing or treating elevated cholesterol levels, atherosclerosis, hyperlipidemia, hypercholesterolemia, cardiovascular events and disease including coronary events and cerebrovascular events, and coronary artery disease and/or cerebrovascular disease in a patient in need thereof.

9. The method of claim 1, wherein the lipid modifying agent is a statin.

10. The method of claim 9, wherein the statin is selected from the group consisting of cerivastatin, atorvastatin, simvastatin, pravastatin, fluvastatin, lovastatin, rosuvastatin, and pitavastatin.

11. The method of claim 9, wherein the statin is present in less than about 10-50 mg/d.

12. A pharmaceutical composition comprising a therapeutically effective amount of a lipid modifying agent, selected from the group consisting of statins, ezetimibe, fibrates, niacinamide, niacin and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid.

13. The composition of claim 12, wherein the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

14. The composition of claim 12, wherein the fatty acid is an omega 3-fatty acid or a fatty acid that is metabolized to an omega 3-fatty acid.

15. The composition of claim 14, wherein the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

16. The composition of claim 12, wherein the phospholipid composition is a compound of Formula (I): 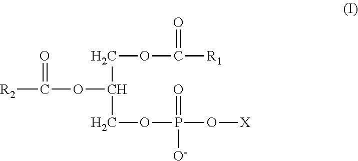
wherein R1 and R2, each together with the respective carboxyl groups to which each is attached, each independently represent a docosahexaenoic acid (DHA) or an eicosapentaenoic acid (EPA) residue, and X is —CH₂CH₂NH₃, —CH₂CH₂N(CH₃)₃, or

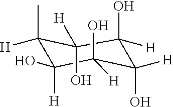

17. A pharmaceutical composition comprising a therapeutically effective amount of a lipid modifying agent, selected from the group consisting of statins, ezetimibe, fibrates, niacinamide, niacin and a therapeutically effective amount of a phospholipid composition comprising a glycerol backbone wherein one of the sn-1 and sn-2 positions is selected from a docosahexaenoic acid (DHA) or an eicosapentaenoic acid (EPA) residue and the other sn-1 and sn-2 position is a fatty acid.

18. The composition of claim 17, wherein the fatty acid is selected from the group consisting of: myristic (14:0); myristoleic (14:1); pentadecanoic (15:0); palmitic (16:0); palmitoleic (16:1); stearic (18:0); oleic (18:1); linoleic (18:2 (n-6)); gamma-linolenic (GLA) (18:3 (n-6)); alpha-linolenic (ALA) (18:3 (n-3)); octadecatetraenoic (OTA) (18:4 (n-3)); arachidic (20:0); cis-11-eicosenoic (20:1); eicosadienoic (20:2 (n-6)); methyl ETA (20:3 (n-6)); arachidonic (20:4 (n-6)); homo-γ-linolenic (20:3 (n-3)); eicosatetraenoic (20:4 (n-3)); EPA (20:5 (n-3)); behenic (22:0); erucic (22:1); docosadienoic (C22:2 (n-6)); adrenic (C22:4 (n-6)); methyl DPA (22:5 (n-6)); docosapentaenoic (DPA) (22:5 (n-3)); DHA (22:6 (n-3)); lignoceric (24:0); and nervonic (24:1).

19. The composition of claim 17, wherein the fatty acid is an omega 3-fatty acid or a fatty acid that is metabolized to an omega 3-fatty acid.

20. The composition of claim 19, wherein the omega 3-fatty acid or a fatty acid that is metabolized to an omega 3-fatty acid is selected from the group consisting of: hexadecatrienoic acid (HTA) (16:3 (n-3)); alpha-linolenic acid (ALA) (18:3 (n-3)); stearidonic acid (SDA) (18:4 (n-3)); eicosatrienoic acid (ETE) (20:3 (n-3)); eicosatetraenoic acid (ETA) (20:4 (n-3)); eicosapentaenoic acid (EPA) (20:5 (n-3)); heneicosapentaenoic acid (HPA) (21:5 (n-3)); docosapentaenoic acid (DPA) (22:5 (n-3)); docosahexaenoic acid (DHA (22:6 (n-3)); tetracosapentaenoic acid (24:5 (n-3)); and tetracosahexaenoic acid (24:6 (n-3)).

21. The composition of claim 17, wherein the phospholipid composition is a compound of Formula (I): 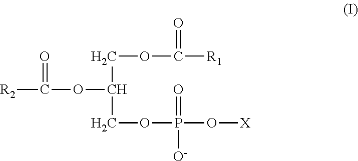
wherein R1 and R2, each together with the respective carboxyl groups to which each is attached, each independently represent a docosahexaenoic acid (DHA) or an eicosapentaenoic acid (EPA) residue, and X is —CH₂CH₂NH₃, —CH₂CH₂N(CH₃)₃, or [chemical structure]
